(19) United States
(12) Patent Application Publication
(10) Pub. No.: US 2014/0100409 A1
(43) Pub. Date: Apr. 10, 2014

(54) Targeted Nuclear Fusion Tumor Therapy

(71) Applicant: Milford Burton Kime, Cambridge, MA (US)

(72) Inventor: Milford Burton Kime, Cambridge, MA (US)

(21) Appl. No.: 14/031,715

(22) Filed: Sep. 19, 2013

Related U.S. Application Data
(60) Provisional application No. 61/711,446, filed on Oct. 9, 2012.

(51) Int. Cl. A61N 5/10

(52) U.S. Cl. CPC A61N 5/1001; A61N 5/1039; A61N 5/1077; A61N 2005/1087
USPC 600/4

(57) ABSTRACT

A method and apparatus for treating a tumor in a patient. In one embodiment, the method comprises the steps of: dosing the patient with a substituted tumor-specific agent and irradiating the tumor with energetic ions, the ions having sufficient energy to penetrate into the tumor and cause nuclear fusion reactions, but not to pass through the tumor. In one embodiment, the substituted tumor-specific agent incorporates deuterium. In this embodiment, the energetic ions are tritium nuclei. In this embodiment, the apparatus comprises a tritium ion source, an accelerator producing an ion beam, a scanning mechanism, and a plurality of neutron detectors positioned adjacent to the patient. The tritium ion beam is programmed to traverse the site of the tumor with tritium ions having energy sufficient to penetrate into the tumor and cause nuclear fusion reactions, but not pass through the tumor.

### CROSS-REFERENCE TO RELATED APPLICATIONS

This application claims priority to and the benefit of U.S. provisional patent application 61/711,446, filed on Oct. 9, 2012, the entire contents of which are hereby incorporated by reference herein.

### FIELD OF THE INVENTION

The invention generally relates to a method and apparatus for radiation oncology and the treatment of tumors. More specifically, the invention relates to specially modified tumor specific agents TSA (e.g. monoclonal antibodies, growth proteins, viral vectors, etc.) to image and treat patient's tumors in a diagnostic/therapeutic scanner.

### BACKGROUND OF THE INVENTION

The field of radiation oncology is rich in tumor therapy procedures. One of the most frequently used is fractionated photon therapy. This procedure utilizes gamma rays produced by the Bremstrahlung process or decay of isotopes like Co60. Beams of gamma rays are collimated to conform to tumor profiles and multiple angle beams are used to minimize the amount of radiation ionization at the surface of the patient. This is necessary because the ionization profile for gamma rays is an exponential with the highest levels at entry. The dose must be fractionated over time to allow healthy cells exposed by the gamma rays to recover from DNA damage. Often as many as 30 treatments are required.

In order to avoid the problems of photon therapy, proton beams have been employed. Protons have charge, and thus can be formed into tightly controlled beams. In addition, the ionization is mostly confined to the well-known Bragg curve peak near the end of range. This allows greater control of dose to the tumor and reduces dose to the intervening healthy cells. Unfortunately, the procedure also requires fractionation, and in some cases the difference in outcomes relative to photon therapy does not seem to warrant the greater expense.

A procedure known as Brachytherapy has been used in cases like prostate cancer. This procedure involves the surgical insertion of radioactive “seeds” directly into the tumor and thus is, in a sense, tumor specific. Unfortunately, it is an invasive procedure.

The most interesting procedures, that are still mostly experimental, are neutron boron-ten irradiation and the bonding of radioactive isotopes to tumor-specific agents. Neutron boron-ten therapy requires bonding of the boron-ten to a tumor-specific agent and followed by in situ activation by neutron beams. Unfortunately, neutrons can cause even greater damage to healthy cells than photons, and like photons, have an exponential-type ionization curve that decreases with depth. Tumor-specific agents with radioactive nuclei attached must be used carefully as they can errantly attach to healthy cells that have cell surface characteristics similar to cancer cells, particularly if the healthy cells have high mitotic rates.

What is needed is a method and apparatus that incorporates the attributes of the current procedures, such as being non-invasive and highly targetable, while avoiding the issues of dose fractionation and healthy cell toxicity.

The present invention addresses these needs.

### SUMMARY OF THE INVENTION

In one aspect, the invention relates to a method for treating a tumor in a patient comprising the steps of: injecting (or orally dosing) the patient with a substituted tumor-specific agent and irradiating the tumor with energetic ions, the ions having sufficient energy to penetrate into the tumor and cause a nuclear fusion reaction, but not to pass through the tumor. In another embodiment, the method includes determining if the dosing of the patient meets predetermined treatment requirements by measuring the concentration of deuterium or tritium in the tumor mass using MRI spectral analysis. In another embodiment, the substituted tumor-specific agent incorporates tritium. In one embodiment, the substituted tumor-specific agent incorporates deuterium. In another embodiment, the energetic ions are tritium nuclei. In still yet another embodiment, the energetic ions are deuterium nuclei.

In another aspect, the invention relates to an apparatus for treating a tumor in a patient by dosing the patient with a deuterium-substituted tumor-specific agent. The apparatus in one embodiment includes a tritium ion source, a beam generating accelerator, a scanning mechanism, and a plurality of neutron detectors positioned adjacent to the patient, wherein the tritium ion beam scans the site of the tumor with tritium ions having an energy sufficient to penetrate into the tumor and cause a nuclear fusion reaction, but not pass through the tumor. In one embodiment, the tritium ion beam apparatus includes a tritium ionization chamber and an accelerator.

In still yet another aspect, the invention relates to an apparatus for treating a tumor in a patient by dosing the patient with a tritium-substituted tumor-specific agent. The apparatus in one embodiment includes a deuterium ion source, a beam generating accelerator, a scanning mechanism, and a plurality of neutron detectors positioned adjacent to the patient, wherein the deuterium ion beam scans the site of the tumor with deuterium ions having an energy sufficient to penetrate into the tumor and cause a nuclear fusion reaction, but not pass through the tumor. In one embodiment, the deuterium ion beam apparatus includes a deuterium ionization chamber, and an accelerator.

In yet another aspect, the invention relates to a medicament for use with nuclear fusion therapy. In one embodiment, the medicament includes a tumor-specific agent in which the hydrogen has been partially or completely substituted with deuterium. In yet another embodiment, the medicament includes a tumor-specific agent in which the hydrogen has been partially or completely substituted with tritium. In another embodiment, the tumor-specific agents are selected from the group of monoclonal antibodies, small molecules, viral vectors, organic nanoparticles and growth proteins.

### BRIEF DESCRIPTION OF THE DRAWING

The objects and features of the invention can be better understood with reference to the drawing described below. The drawing is not necessarily drawn to scale; emphasis is instead being placed on illustrating the principles of the invention. In the drawing, numerals are used to indicate specific parts throughout the various views. The drawing associated with the disclosure is addressed on an individual basis within the disclosure as it is introduced.

FIG. 1 is a highly schematic diagram of an irradiation apparatus constructed in accordance with the invention.

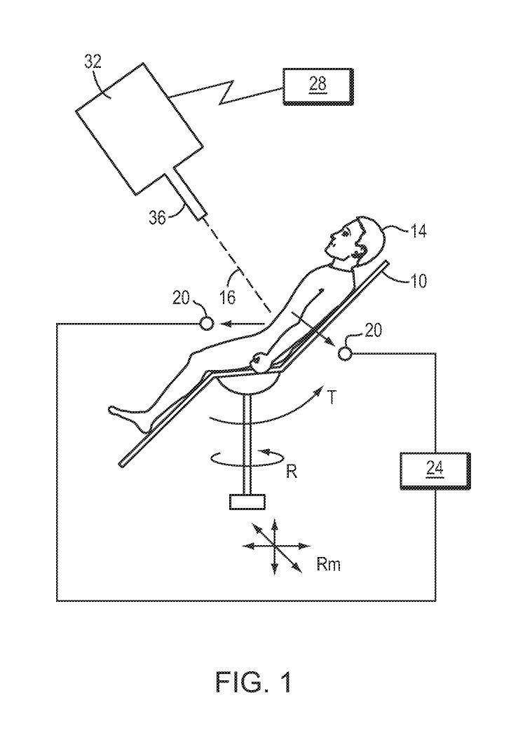

### DESCRIPTION OF AN EMBODIMENT OF THE INVENTION

In brief overview, the invention makes use of the fusion of a deuterium nucleus and a tritium nucleus to form an unstable helium-five nucleus, which decays to form an energetic neutron (14.1 MeV) and an energetic (3.5 MeV) alpha particle (helium four nucleus). Tumor specific agents (TSAs) that attach to the tumor are modified to include deuterium rather than hydrogen in their composition. When the deuterium containing TSA is irradiated with a tritium beam, the deuterium and tritium reaction components are converted to an alpha particle and neutron. The alpha particle imparts energy to the tumor cell, destroying it. In another embodiment, tritium may be incorporated into the TSAs and the tritiated-TSAs irradiated by a deuterium ion beam.

Like proton therapy, this procedure exploits the Bragg peak dosage concentration which other procedures, such as neutron boron-ten therapy, do not. Also, the particles used in irradiation are not neutrons, and the neutrons emitted from the deuterium-tritium fusion reactions are not as numerous as those used in the activation of boron-ten. As such, the neutrons produced will not have the same damaging effect on healthy cells.

Unfortunately, the relatively high energy of the neutrons formed is something of a double-edged sword. Without this energy, the neutrons would not escape the patient, and thus no diagnostic imaging and therapy guidance would be possible. However, the initial collisions of the neutrons near the point of origin produce energetic collision ions, and some of these will dissipate their energy in adjacent anatomy outside the tumor volume. Fortunately, the neutron detector arrays can function as real time dosimeters. If the resulting neutron dose to the anatomy adjacent the tumor becomes a problem, then the beam energy can be adjusted to place the end of range outside the tumor, and the therapy can continue using the Bragg peak ionization method. In this case, tritium ions will not undergo fusion reactions, but rather are implanted in the margins around the tumor, thus treating the margins with a Brachytherapy-like delayed procedure.

In using tritium, either as a TSA label or as the ionization beam, it must be remembered that tritium is radioactive and thus must be handled with care. Fortunately, tritium in a gas molecule form (such as used in the feed stock for an ionization chamber) is ten thousand times less hazardous than in an aqueous molecular form (which is often the case during leaks from nuclear power plants) and the Department of Energy has developed procedures for handling various tritiated forms.

### Tumor Specific Agents

The tumor specific agents include benign modifications, both of numerous known and yet-to-be-found agents. TSAs include, but are not limited to, monoclonal antibodies, small molecules, viral vectors, organic nanoparticles and growth proteins. In the deuterated embodiment, the TSA molecules do not have chemo toxins or radioactive nuclei attached. Instead, the modification to a TSA is the simple replacement of the common hydrogen (a.k.a. proteum) found in its molecular structure, with the isotope deuterium. The chemistry for doing this is well known to one skilled in the art. In fact, researchers have succeeded in creating living mice that have a substantial amount of the hydrogen in their cells replaced by deuterium. The deuterium should do very little to modify the chemistry or biology of the TSA. Unlike some therapies exploiting radioactive or chemotoxic TSAs, no chemotoxins or radioactive isotopes are attached to make the TSA oncolytic, so large doses can be administered without fear of damaging healthy cells.

The TSAs that are deuterated come in many forms and concentrate in different parts of tumors. For example, monoclonal antibodies attach to the surface of cells. Small molecule TSAs and viral vectors enter the interior of the cell. Organic nanoparticles become trapped in the vasculature supporting the growth of the tumor. This great variety of tumor specific agents make it possible to achieve high densities of deuterium, thus achieving useful macroscopic cross sections for fusion reactions.

Conversely, TSA that are tritiated require more careful handling due to their radioactive nature. However, it is important to realize that the inherent radioactivity of the tritiated TSA is not intended as the primary treatment of the tumor. Primarily, it is the fusion of the deuterated ions with the tritium nuclei that generate the high energy particles that destroy the tumor cells.

### Scanner

A scanning approach that is easily implemented in existing accelerator facilities produces a scan by orienting and traversing the patient rather than the beam. It is analogous to the numerically controlled end mills used to machine precise mechanical parts with complex surface contours.

In one embodiment, a chair-like frame 10 is used to immobilize the patient 14. The patient can be seated facing away from or toward the frame depending on the site of the tumor. The frame 10 can be tilted (arrow T) at various angles thru 90 degrees from vertical to horizontal and rotated 360 degrees about a vertical axis (arrow R), thus aligning the ion beam trajectory 16 with the tumor site from multiple arbitrary orientations. Mounting points on the frame allow the placement of neutron detector arrays 20 (only two shown for clarity) near the patient's body. Signals from the neutron detectors are processed by a processor 24 to provide imaging displays and dosage calculations. The frame 10 is mounted on a traversing table that can both position the patient and produce rectilinear motion (arrows RM) in all three dimensions, thus creating a scan which can envelop the tumor volume by varying the beam energy and placement. In the case of incapacitated patients, the chair-like frame 10 can be replaced by a bed-like frame that can be tilted about both its long and narrow axes. This requires beam optics that can orient the beam from a nearly vertical direction.

An equivalent scanning mechanism (similar to that used in proton therapy) that is somewhat more elegant, but more complex, leaves the patient motionless, reclined on a table, and traverses the beam by using a gantry similar to the C-arms used in fluoroscopy exams, exploiting magnetic and electrostatic beam-guiding techniques well known in the art of ion beam optics.

In use, in one embodiment, a tritium gas 28 is the feedstock to an ionization chamber 32 which produces tritium ions. The resulting tritium ions are then accelerated by an ion accelerator 36 to produce the tritium ion beam 16. In another embodiment, in which a deuterium ion beam is used, deuterium gas 28 is used in the ionization chamber 32.

### The Therapy Process

In the imaging phase of the treatment, in one embodiment, the tritium ion beam is scanned at low intensity in areas where a tumor, labeled with TSAs, is indicated by conventional radiology techniques. In particular, MRI spectral analysis can be useful in assessing the concentration of deuterium or tritium in the tumor mass, and thus whether the dosing of the patient has been successful. The beam energy is selected so that the end of range of penetration into the patient is within the tumor site. This assures that the tritium ion energy will be at or near the peak of the absorption cross-section curve for the fusion reaction of deuterium and tritium nuclei.

When a fusion reaction occurs between a tritium ion and a deuterium nucleus in the TSA, the products of the reaction are an 3.5 MeV alpha particle and a 14.1 Mev neutron. Some of the neutrons thus produced escape the patient and can be detected by the neutron detector array 20 placed around the patient. The detection of neutrons indicates the presence of deuterated TSA and from the resulting neutron detection, the tumor mass can be surveyed and an image can be displayed.

The therapy phase involves three processes. The alpha particles created by fusion reactions will necrotize the cells where the TSA is located due to their short range and 3.5 Mev energy. Beam direction can be chosen so that the peak of the Bragg ionization curve remains in the tumor mass and also in margins around the tumor to assure complete destruction. This second process is similar to that used in proton beam therapy. Finally, when the deuterium in the tumor mass becomes depleted, the tritium ions that do not undergo a fusion reaction will still become implanted in the tumor. Tritium decays by emission of a beta particle, so over time there will be a continuing dosing of the tumor volume with short range ionizing radiation, thus decreasing the probability of a recurrence.

This embodiment of therapy has implications for cost. Because the tritium ions and their radiation decay products are precisely targeted and geometrically dispersed as well as time delayed, there is little need for fractionating the total dose. This allows more patients to be treated per scanner and therefore lower cost per patient. This is important as versions of the scanners capable of treating deep-sited tumors may prove to be expensive.

It is important to note that this therapy is not appropriate for all tumors. It is best suited for the small isolated tumors of organs such as the prostate, breast, kidneys, etc. that are the manifestation of focal cancer. In the case of larger tumors, the standard protocol of surgical resection followed by chemotherapy shrinking and finally radiation therapy should be followed. One group of patients who may benefit from this somewhat complicated procedure is those who have proven to be very difficult to treat. Chief among these are patients with a glial blastoma multiforme (GBM). This unfortunately common primary brain tumor is driven by angiogenesis which supports its aggressive growth. Fortunately, small molecule tumor-specific agents and targeting organic nanoparticles have the ability to cross the blood-brain barrier and each presents a targeted path to attacking GBM's. The small molecule TSA's concentrate deep in the tumor cells and the organic nanoparticles concentrate in the vasculature supporting the tumor growth. Dosing and imaging the patient sequentially with first small molecule TSA's and then targeting organic nanoparticles will produce two images, one of the tumor mass and the second a combination of the mass and the vasculature supporting its growth. Subtracting the first from the second will give isolated views of each, and thus inform the oncologist as how to best partition the therapy between angiogenesis inhibition and tumor necrosis.

It is to be understood that the FIGURE and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the FIGURE are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Furthermore, whereas particular embodiments of the invention have been described herein for the purpose of illustrating the invention and not for the purpose of limiting the same, it will be appreciated by those of ordinary skill in the art that numerous variations of the details, materials and arrangement of elements, steps, structures, and/or parts may be made within the principle and scope of the invention without departing from the invention as described in the claims.

Variations, modification, and other implementations of what is described herein will occur to those of ordinary skill in the art without departing from the spirit and scope of the invention as claimed. Accordingly, the invention is to be defined not by the preceding illustrative description, but instead by the spirit and scope of the following claims.

What is claimed is:

1. A method for treating a tumor in a patient comprising the steps of:
dosing the patient with a substituted tumor specific agent; and
irradiating the tumor with energetic ions the ions having sufficient energy to penetrate into the tumor and cause a nuclear fusion reaction, but not to pass through the tumor.

2. The method of claim 1 wherein the substituted tumor specific agent incorporates deuterium.

3. The method of claim 1 wherein the energetic ions are tritium nuclei.

4. The method of claim 1 wherein the substituted tumor specific agent incorporates tritium.

5. The method of claim 4 wherein the energetic ions are deuterium nuclei.

6. The method of claim 1 further comprising the step of determining if the dosing of the patient meets predetermined treatment requirements by measuring the concentration of deuterium or tritium in the tumor mass using MRI spectral analysis.

7. An apparatus for treating a tumor in a patient dosed with a deuterium substituted tumor specific agent, the apparatus comprising:
a tritium ion source;
a tritium ion beam generating accelerator;
a scanning mechanism for scanning the tritium ion beam; and
a plurality of neutron detectors positioned adjacent to the patient,
wherein the tritium ion beam is caused to scan the site of the tumor with tritium ions having an energy sufficient to penetrate into the tumor and cause a nuclear fusion reaction but not pass through the tumor.

8. An apparatus for treating a tumor in a patient dosed with a tritium substituted tumor specific agent, the apparatus comprising:
a deuterium ion source;
a deuterium ion beam generating accelerator;
a scanning mechanism for scanning the deuterium ion beam; and
a plurality of neutron detectors positioned adjacent to the patient,
wherein the deuterium ion beam is caused to scan the site of the tumor with deuterium ions having an energy sufficient to penetrate into the tumor and cause a nuclear fusion reaction but not pass through the tumor.

9. A medicament for use with nuclear fusion therapy comprising a tumor specific agent in which the hydrogen has been partially or completely substituted with deuterium.

10. The medicament of claim 9 wherein the tumor specific agents are selected from the group of monoclonal antibodies, small molecules, viral vectors, nanoparticles and growth proteins.

11. A medicament for use with nuclear fusion therapy comprising a tumor specific agent in which the hydrogen has been partially or completely substituted with tritium.

12. The medicament of claim 11 wherein the tumor specific agents are selected from the group of monoclonal antibodies, small molecules, viral vectors, organic nanoparticles and growth proteins.
